(19) United States
(12) Patent Application Publication
(10) Pub. No.: US 2016/0157985 A1
(43) Pub. Date: Jun. 9, 2016

(54) Apparatus and Methods for Removing an Obstruction from a Bodily Duct of a Patient

(71) Applicants: Stryker Corporation, a Michigan Corporation, Kalamazoo, MI (US); Stryker European Holdings I, LLC, Kalamazoo, MI (US)

(72) Inventors: Justin T. Vo, San Jose, CA (US); Hancun Chen, San Ramon, CA (US)

(21) Appl. No.: 14/950,203

(22) Filed: Nov. 24, 2015

Related U.S. Application Data
(60) Provisional application No. 62/087,005, filed on Dec. 3, 2014.

(51) Int. Cl. A61F 2/01; A61B 17/221

(52) U.S. Cl. CPC A61F 2/01; A61F 2/013; A61B 17/221; A61F 2002/016; A61B 2017/2212; A61B 2017/2215

(57) ABSTRACT

A bodily duct obstruction retrieval device that includes a self-expandable member that is capable of transitioning between unexpanded and expanded states. According to some implementations the inner wall surface of the self-expandable member has one or more features protruding therefrom that are adapted to engage the obstruction as the self-expandable member transition from the expanded state toward the unexpanded state. According to other implementations the outer wall surface of the self-expandable member also has one or more features protruding therefrom that are adapted to engage the obstruction as the self-expandable member transition from the unexpanded state toward the expanded state.

### TECHNICAL FIELD

This application relates to apparatus and methods for removing obstructions, such as blood clots, within the vasculature or other internal bodily ducts of a patient.

### BACKGROUND

Devices for capturing and removing blockages within the vasculature or other internal bodily ducts of a patient have been developed. Some of these devices comprise self-expanding stent-like prostheses that are attached to the end of an elongate wire. The prostheses are typically delivered via a delivery catheter in an unexpanded state to the sof the blockage. Capture of the blockage is typically achieved by retracting the delivery catheter so that the struts of the prosthesis expands into the blockage. Once captured, removal of the obstruction is typically achieved by retracting the prosthesis by use of the elongate wire to a position outside the patient. A problem with many devices is that the prosthesis fails to adequately integrate into the blockage, resulting in only a partial capture of the blockage. Another problem is that even when properly captured the prosthesis tends to lack the requisite gripping properties to maintain the blockage on the prosthesis as it is removed from the patient. As a result of these problems, removal of a blockage generally requires multiple deployments of the prosthesis to effectuate proper removal. These multiple deployments increase procedure time and costs, and can also lead to portions of the blockage being dislodged and passing downstream the site of the blockage. What is needed are devices that are capable of more optimally engaging and retaining a blockage in the course of the blockage being removed from the patient.

### SUMMARY OF THE DISCLOSURE

According to some implementation a bodily duct obstruction retrieval device is provided that comprises a self-expandable member that is capable of transitioning between unexpanded and expanded states, the self-expandable member having a wall formed by a plurality of interconnected struts, the interconnected struts forming a plurality of cell structures in the wall, the interconnected struts forming the wall having an inner surface and an outer surface, at least some of the interconnected struts having spaced-apart protruding elements positioned along a length of the inner surface, the protruding elements being separated by gaps and configured to engage the obstruction as the self-expandable member transition from the expanded state toward the unexpanded state, the self-expandable member having a proximal end and a distal end.

According to other implementations the outer wall surface of the self-expandable member also has protruding elements that are configured to engage the obstruction as the self-expandable member transition from the unexpanded state toward the expanded state.

According to some implementations the protruding elements comprise teeth that are substantially uniformly dispersed along a length of the inner and/or outer surface of the interconnect struts.

According to other implementations at least some or all of the struts of the self-expandable member are devoid of protruding elements and are instead wound about by a wire or filament having protruding elements, the protruding elements on the wire or filament configured to engage an obstruction as the self-expandable member transition between the expanded and unexpanded states.

### BRIEF DESCRIPTION OF THE DRAWINGS

FIGS. 1A and 1B illustrate exemplary bodily duct obstruction retrieval devices.

FIGS. 2A-C illustrate a method of deploying a bodily duct obstruction retrieval device according to one implementation.

FIG. 3 is a two-dimensional plane view of the bodily duct obstruction retrieval device of FIG. 1A.

FIGS. 4A-E illustrate cross-section views of struts that form an expandable member of a bodily duct obstruction retrieval device according to some implementations.

FIGS. 5A-C show exemplary strut locations with which the exemplary features of FIGS. 4A-E may reside.

FIG. 6 illustrates a strut segment of varying width with the portion of the strut containing the protruding elements having an enhanced width dimension.

FIG. 7A shows a tube from which an expandable member of an bodily duct obstruction retrieval device may be laser cut, the inner wall of the tube having micro-channels formed therein.

FIG. 7B shows a tube from which an expandable member of a bodily duct obstruction retrieval device may be laser cut, the inner and outer wall of the tube having micro-channels formed therein.

FIGS. 8A and 8B illustrates a method of forming a strut with protruding elements according to one implementation.

FIGS. 9A and 9B illustrates a method of forming a strut with protruding elements according to another implementation.

FIG. 10 illustrates a two-dimensional plane view of a bodily duct obstruction retrieval device according to another implementation.

### DETAILED DESCRIPTION

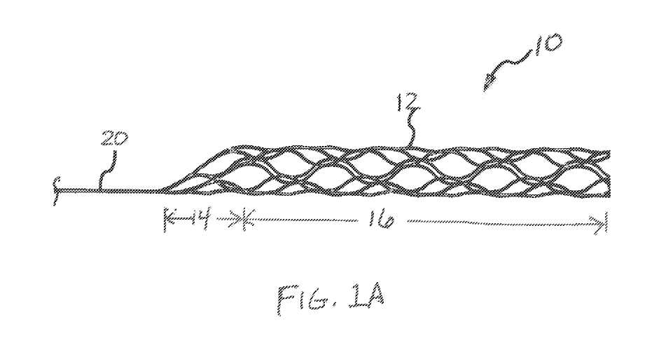

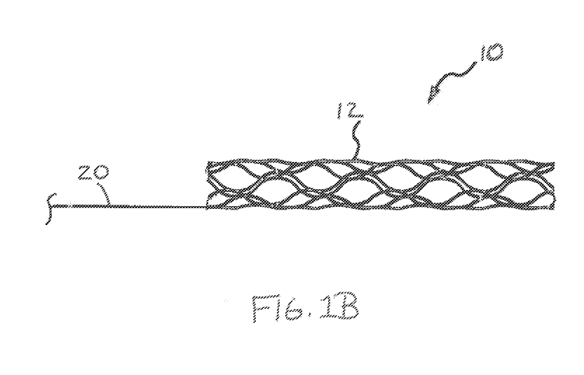

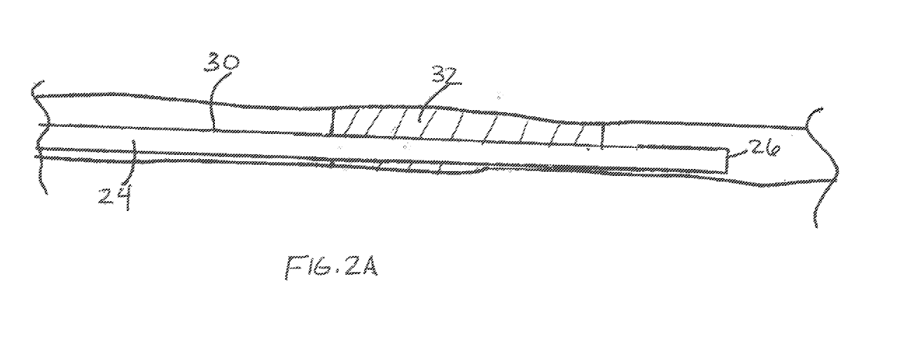

FIGS. 1A and 1B illustrate devices 10 useful for removing obstructions from the bodily duct of a patient, such as, for example, for removing blood clots in the neurovasculature. In the example of FIG. 1A the device 10 includes a self-expandable member 12 having an elongate wire 20 connected to a proximal end thereof. The self-expandable member 12 includes a proximal taper portion 14 and a cylindrical main body portion 16. The device of FIG. 1B is similar to that of FIG. 1A, but is devoid of a proximal taper portion. The self-expandable member 12 of each of the devices is capable of transitioning between unexpanded and expanded states. FIGS. 1A and 1B illustrate the self-expandable member 12 in a fully expanded state. In use, the degree to which the self-expandable members 12 expand is contingent on the biological profile at the site of the vessel in which it is deployed. According to some implementations the expandable member 12 is made of a shape memory material, such as Nitinol, and may be laser cut from a tube as will be discussed in more detail below. The expandable member 12 may also be fabricated from a laser cut or etched metal sheet that is formed into a substantially cylindrical shape subsequent to the struts being formed in the sheet. In such instances, once the struts have been formed, the opposing ends of the sheet may be welded together.

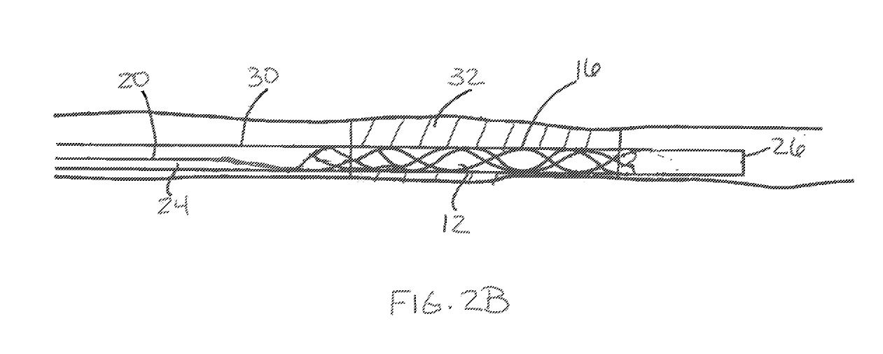

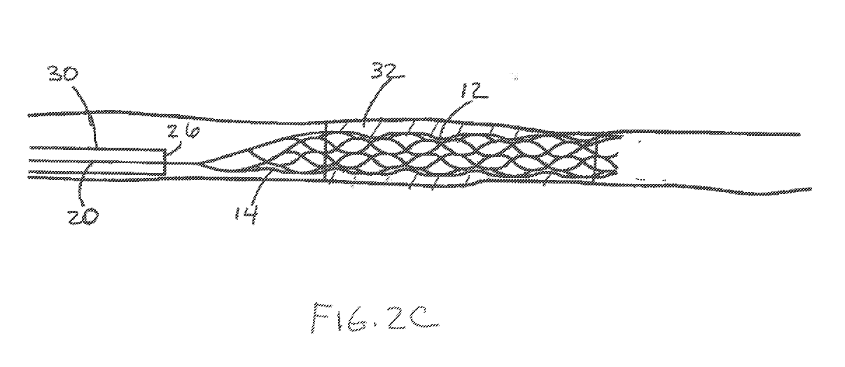

In procedures involving the removal of a blood clot from the neurovasculature, the expandable member 12 is advanced through the tortuous vascular anatomy of a patient to the site of the blood clot in the unexpanded state. FIGS. 2A-C illustrate one manner of delivering and deploying the expandable member 12 at the site of a blood clot 32. As shown in FIG. 2A, a delivery catheter 30 having an inner lumen 24 is advanced to the site of the clot 32 so that its distal end 26 is positioned distal to the clot. After the delivery catheter 30 is in position at the site of the clot 32, the retrieval device 10 is placed into the delivery catheter by introducing the expandable member 12 into a proximal end of the delivery catheter (not shown) and then advancing the expandable member 12 through the lumen 24 of the delivery catheter 30 by applying a pushing force to the elongate flexible wire 20. By the use of radiopaque markings and/or coatings positioned on the delivery catheter 30 and device 10, the expandable member 12 is positioned at or near the distal end 26 of the delivery catheter 30 as shown in FIG. 2B so that the main body portion 16 is longitudinally aligned with the clot 32. Deployment of the expandable member 12 is achieved by proximally withdrawing the delivery catheter 30 while holding the expandable member 12 in a fixed position as shown in FIG. 2C. As the expandable member 12 expands while being deployed from the catheter 30, the struts forming the expandable member integrate into the clot 32 causing a least a portion of the clot to reside within an internal lumen of the expandable member 12. Upon the clot 32 being captured the expandable member 12 is retracted, along with the delivery catheter 30, to a position outside the patient. In some situations the expandable member 12 is first partially retracted to engage with the distal end 26 of the delivery catheter 30 prior to fully retracting the devices from the patient.

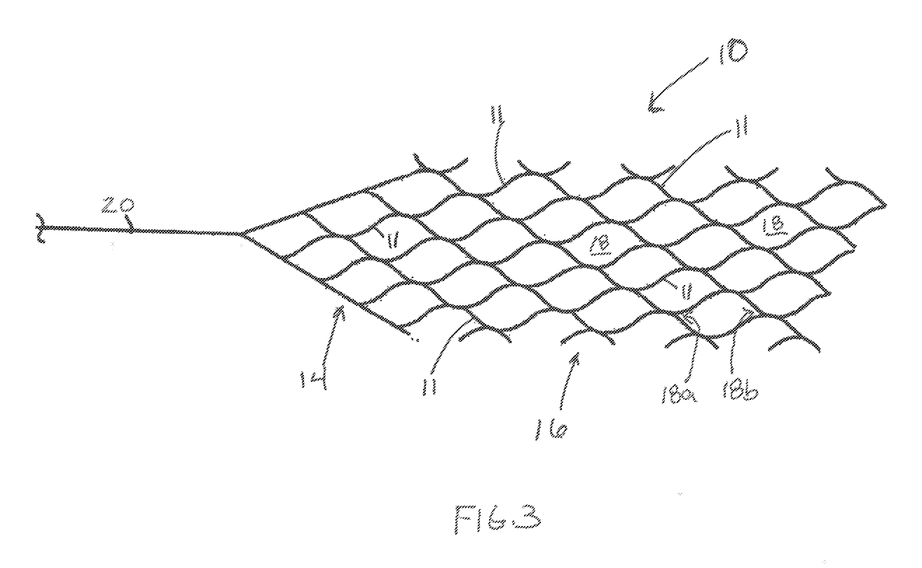

FIG. 3 shows device 10 of FIG. 1A in a two-dimensional plane view as if the expandable member 12 were cut and laid flat on a surface. The wall of the expandable member 12 is formed by a plurality of interconnected struts 11. FIG. 3 illustrates the inner surface of the struts 11. The dimensions of the struts 11 may vary within the expandable member 12 itself and generally comprise a width dimension between 0.0025 to 0.005 inches and a thickness dimension between 0.0025 to 0.0035 inches. The interconnected struts 11 form cell structures 18 that in the implementation of FIG. 3 are arranged in a diagonal fashion around the circumference of the expandable member 12. The cell structures 18 comprise proximal and distal apex regions 18a and 18b, respectively. One advantage associated with such a cell pattern is that withdrawing the expandable member 12 by the application of a pulling force on the proximal elongate flexible wire 20 urges the expandable member 12 to assume a smaller expanded diameter while being withdrawn, thus decreasing the likelihood of injury to the vessel wall. Also, during clot retrieval as the diameter of the expandable member 12 decrease, the wall of the expandable member 12 tends to collapse and pinch down on the clot to increase clot retrieval efficacy. Another advantage is that the cell patterns permit the expandable member 12 to be retracted into the lumen of the delivery catheter after it has been partially or fully deployed. As such, if at any given time it is determined that the expandable member 12 has been partially or fully deployed at an improper location, it may be retracted into the distal end of the delivery catheter and repositioned to the correct location.

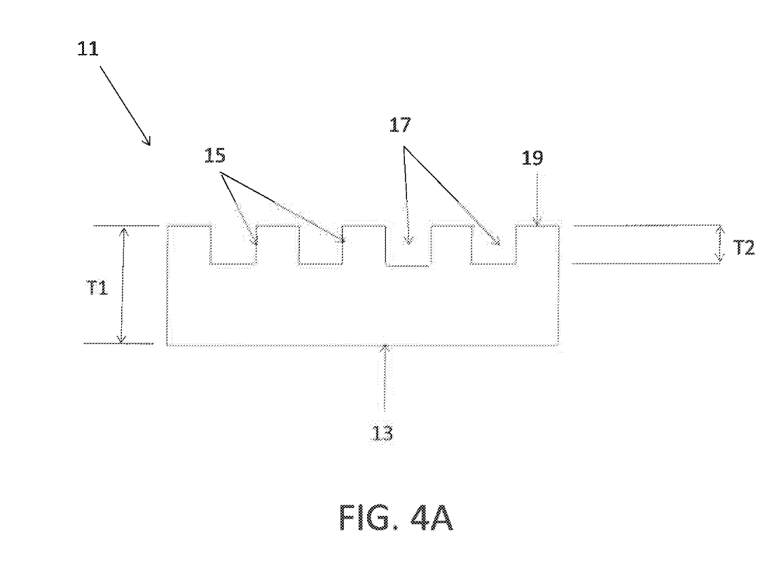

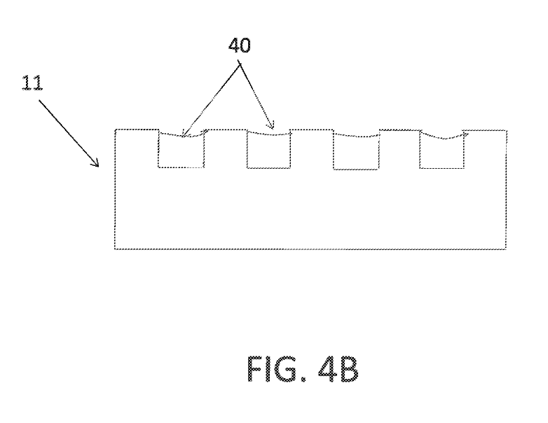

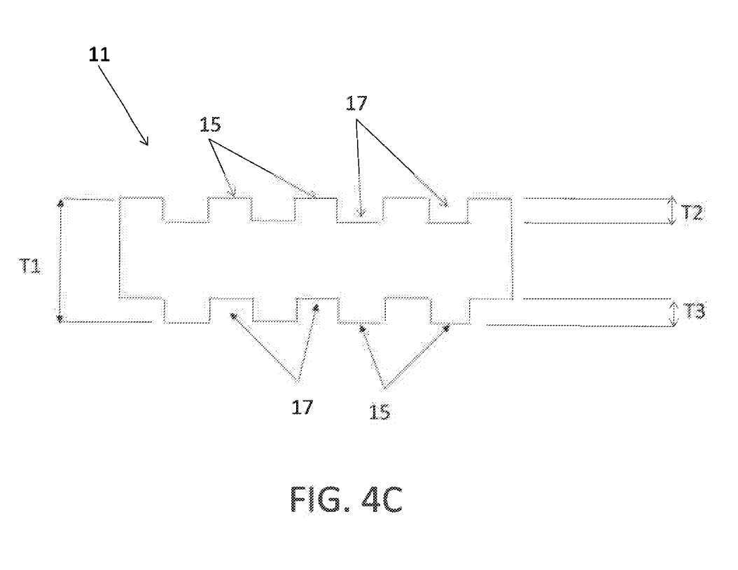

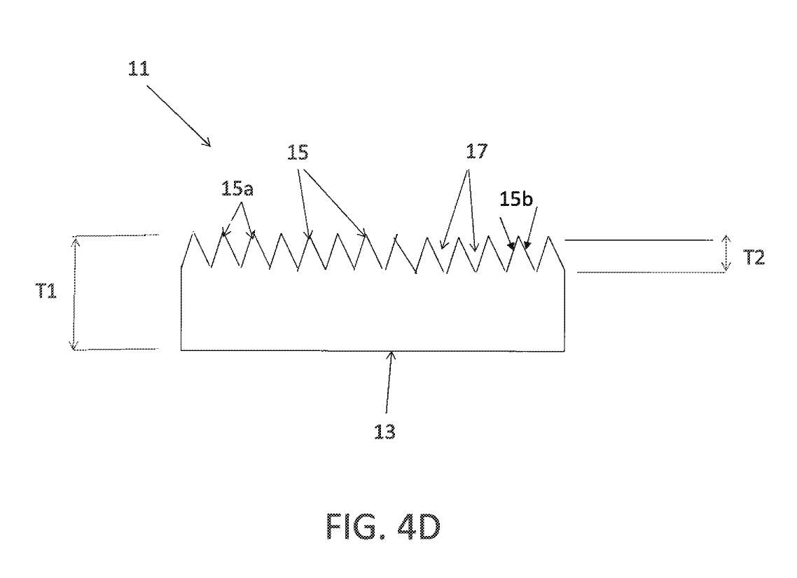

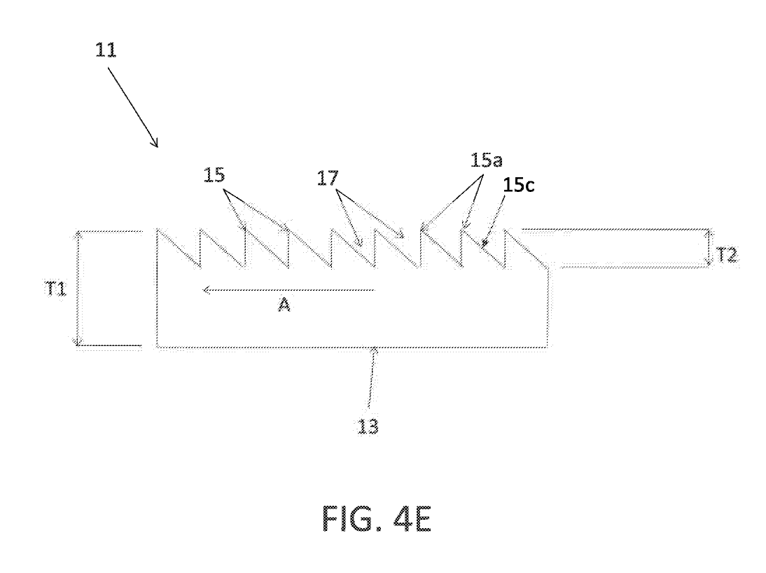

To enhance the expandable member's ability to grip a clot, or other types of obstructions, spaced-apart protruding elements are provided on the inner surface 19 and/or outer surface 13 of the struts 11. FIGS. 4A-E illustrate some examples. Manufacturing methods that may be used to form the protruding elements will be discussed below in conjunction with FIGS. 7 through 9. As a result of the expandable member 12 partially collapsing when a proximal pulling force is applied to elongate wire 20 as discussed above, when the protruding elements are provided on the inner surface 19 of at least some of the struts 11, the protruding elements act on portions of the clot entrapped inside the expandable member 12.

In the implementation of FIG. 4A the inner surface 19 of all or a selected number of struts 11 of the expandable member 12 are provided with protruding elements 15 having a thickness T2 that are separated by gaps 17. The gaps 17 may be in the form of micro-channels formed in the inner surface 19. In the implementation of FIG. 4A the protruding elements 15 are in the form of teeth that are substantially uniformly dispersed along a length of the inner surface 19 of the strut 11. In other implementations the protruding elements are not uniformly dispersed along the length of the strut 11. According to some implementations the width dimension of the protruding elements 15 is substantially the same as the thickness dimension of the strut 11. According to other implementations the width dimension of the protruding elements 15 is between 110% to 150% of the thickness of the strut 11. In yet other implementations the width dimension of the protruding elements 15 is between 50% to 90% of the thickness of the strut 11. According to some implementations, as discussed above, the thickness T1 of the strut 11 may range between 0.0025 to 0.0035 inches. According to some implementations the ratio of the thickness T1 of the strut 11 to the thickness T2 of the protruding elements 15 (T1/T2) is between 2.0 and 5.0. According to other implementations the ratio of the thickness T1 of the strut 11 to the thickness T2 of the protruding elements 15 (T1/T2) is between 5.0 and 10.0. According to other implementations the ratio of the thickness T1 of the strut 11 to the thickness T2 of the protruding elements 15 (T1/T2) is between 10.0 and 15.0. According to other implementations the ratio of the thickness T1 of the strut 11 to the thickness T2 of the protruding elements 15 (T1/T2) is between 15.0 and 60.0. According to some implementations, as shown in FIG. 4B, the gaps 17 may contain a coagulant drug 40 that is releasable into the clot to encourage the clot to stay substantially in-tact during the removal process and to encourage a greater adhesion of the protruding elements 15 with the clot. The use of a coagulant drug 40 is also applicable to each of the implementations of FIGS. 4C-E. That is, the gaps 17 of each of the implementations of FIGS. 4C-E may contain a coagulant drug.

As shown in FIG. 4C, according to some implementations the outer surface 13 of at least some of the struts 11 may also possess protruding elements 15 separated by gaps 17 that are configured to engage the clot as the self-expandable member 12 transitions from the unexpanded state toward the expanded state. According to some implementations the thickness of the protruding elements 15 located along the inner surface 19 of the strut 11 is substantially equal to the thickness of the protruding elements 15 located along the outer surface 13 of the strut 11. That is, thickness dimensions T2 and T3 are substantially the same. According to some implementations the thickness of the protruding elements 15 located along the inner surface 19 of the strut 11 is greater than the thickness of the protruding elements located along the outer surface 13 of the strut 11. That is, thickness dimensions T2 is greater than T3. According to some implementations the thickness of the protruding elements 15 located along the inner surface 19 of the strut 11 is less than the thickness of the protruding elements 15 located along the outer surface 13 of the strut 11. That is, thickness dimensions T2 is less than T3. According to some implementations the thickness T1 of the strut 11 may range between 0.0025 to 0.0035 inches. According to some implementations the ratio of the thickness T1 of the strut 11 to each of the thicknesses T2 and T3 of the protruding elements 15 (T1/T2 and T1/T3) is between 2.0 and 5.0. According to other implementations the ratio of the thickness T1 of the strut 11 to the each of the thicknesses T2 and T3 of the protruding elements 15 (T1/T2 and T1/T3) is between 5.0 and 10.0. According to other implementations the ratio of the thickness T1 of the strut 11 to each of the thicknesses T2 and T3 of the protruding elements 15 (T1/T2 and T1/T3) is between 10.0 and 15.0. According to other implementations the ratio of the thickness T1 of the strut 11 to each of the thicknesses T2 and T3 of the protruding elements 15 (T1/T2 and T1/T3) is between 15.0 and 60.0.

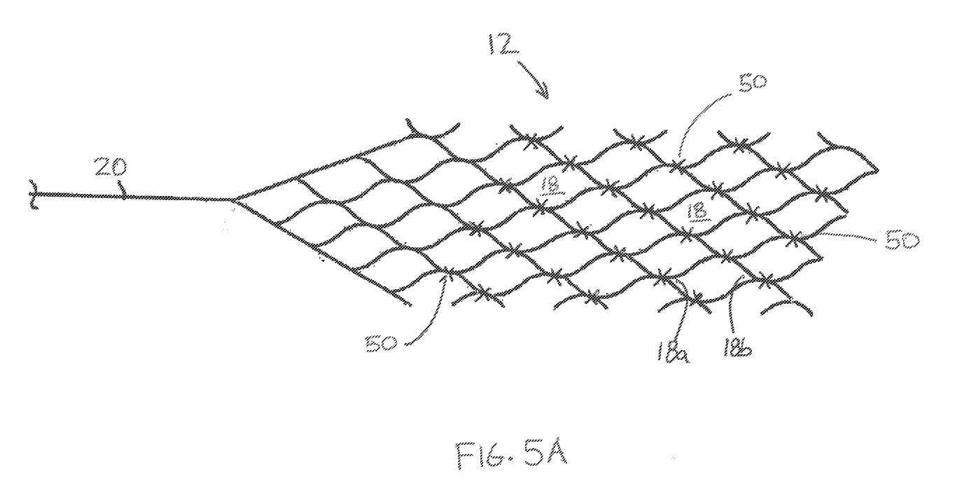

It is important to note that less than all of the struts 11 may possess protruding elements 15, and further that less than all of the length of a strut 11 may possess the protruding elements 15. In the implementation of FIG. 5A, the locations 50 of the protruding elements 15 reside on a portion of the strut 11 where the apexes 18a and 18b of adjoining cell structures 18 meet. An advantage of the implementation of FIG. 5A is that none of the protruding element locations 50 is circumferentially aligned with another. This avoids the protruding elements from interacting with one another when the expandable member 12 assumes the unexpanded state. As illustrated in FIGS. 5B and 5C, the cell structures 18 may possess long struts 11a and short struts 11b. In the implementation of FIG. 5B, the locations 52 of the protruding elements 15 reside on the long struts 11a, whereas in the implementation of FIG. 5C the locations 54 of the protruding elements 15 reside on the short struts 11b. According to some implementations the struts 11 containing the protruding elements 15 have a width dimension that is greater than the width dimension of the other struts not containing protruding elements in the main body portion 16 of the self-expandable member 12.

According to other implementations a substantial portion or all of the inner wall of the main body portion 16 of the self-expandable member 12 possess protruding elements 15.

FIG. 4D illustrates another implementation wherein the protruding elements 15 are formed and arranged in a saw-tooth configuration along the inner surface 19 of the strut 11. The protruding elements 15 are separated by gaps 17 and possess pointed apexes 15a. The pointed apexes 15a reduce the initial contact surface area of the protruding elements 15 with the clot, thus enhancing the ability of the protruding elements 15 to penetrate the clot upon initial contact therewith. Like the implementation of FIG. 4C, the outer surface 13 of the strut 11 may also possess protruding elements 15 arranged in a saw-tooth configuration similar to that of the inner surface 19. According to some implementations the base width dimension of the protruding elements 15 is substantially the same as the thickness dimension of the strut 11. According to other implementations the base width dimension of the protruding elements 15 is between 110% to 150% of the thickness of the strut 11. In yet other implementations the base width dimension of the protruding elements 15 is between 50% to 90% of the thickness of the strut 11. According to some implementations the flanks 15b of the protruding elements 15 have an angle of between 15 and 150 degrees to one another. According to some implementations the thickness T1 of the strut 11 may range between 0.0025 to 0.0035 inches. According to some implementations the ratio of the thickness T1 of the strut 11 to the thickness T2 of the protruding elements 15 (T1/T2) is between 2.0 and 5.0. According to other implementations the ratio of the thickness T1 of the strut 11 to the thickness T2 of the protruding elements 15 (T1/T2) is between 5.0 and 10.0. According to other implementations the ratio of the thickness T1 of the strut 11 to the thickness T2 of the protruding elements 15 (T1/T2) is between 10.0 and 15.0. According to other implementations the ratio of the thickness T1 of the strut 11 to the thickness T2 of the protruding elements 15 (T1/T2) is between 15.0 and 60.0.

FIG. 4E illustrates an implementation similar to that of FIG. 4D with the protruding elements 15 formed and arranged in a saw-tooth configuration along the inner surface 19 of the strut 11. The protruding elements 15 are separated by gaps 17 and possess pointed apexes 15a. As shown in FIG. 4E, the apexes 15a point in a direction A toward the proximal end of the self-expandable member 12. According to some implementations the trailing flank 15c of the protruding elements has an angular orientation of between 10 and 80 degrees with respect to the longitudinal axis of the strut 11. An advantage associated with this sloping orientation of the protruding elements 15 is that it offers less resistance to the distal advancement of the self-expandable member 12 through the obstruction/clot when a pushing force is applied to the proximal end of the self-expandable member by the elongate wire 20. In addition, the sloping orientation of the protruding elements 15 toward the proximal end of the self-expandable member 12 offers an enhanced resistance to the proximal advancement of the self-expandable member 12 through the obstruction/clot when a pulling force is applied to the proximal end of the self-expandable member by the elongate wire 20. This enhanced resistance to the proximal advancement of the self-expandable member 12 through the obstruction/clot improves the self-expandable member's ability to maintain engagement with the obstruction/clot during the process of removing the obstruction/clot from the patient. That is, the obstruction/clot will have less of a tendency to slip off the self-expandable member 12 as the self-expandable member is retracted through the vasculature of the patient. According to some implementations the outer surface 13 of at least some of the struts 11 may also possess protruding elements 15 that slope toward the proximal end of the self-expandable member 12 that are configured to engage the obstruction/clot as the self-expandable member 12 transitions from the unexpanded state toward the expanded state.

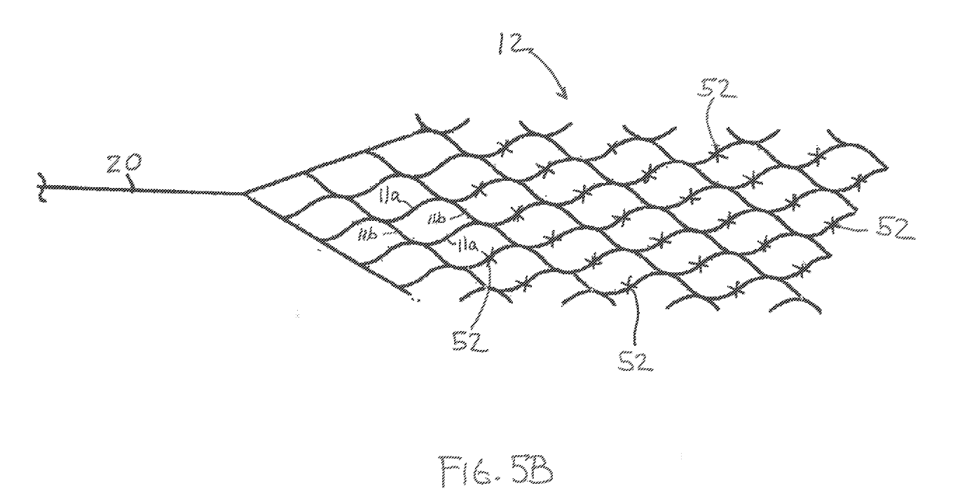

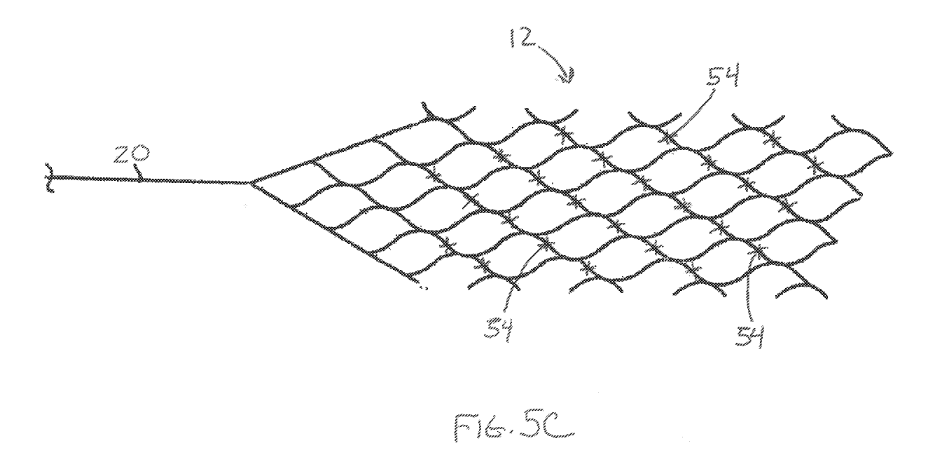

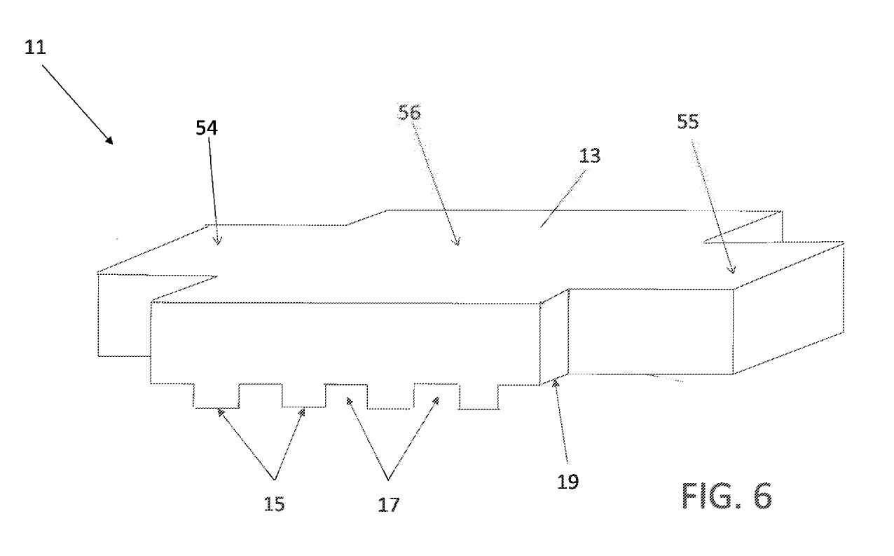

FIG. 6 illustrates a strut 11 according to one implementation. The strut 11 has a central region 56 disposed between proximal and distal regions 54 and 55, respectively. As shown in FIG. 6, the central region 56 has a first width dimension that is greater than the second width dimension of each of the proximal and distal end regions 54 and 55. The protruding elements 15 and gaps 17 being disposed along the inner surface 19 of the strut 11 only within the central region 56. According to some implementations the ratio of the first width dimension to the second width dimension is between 1.1 and 1.5. Further, as with the implementations previously disclosed, the outer surface 13 of the strut 11 may also contain protruding elements 15 and gaps 17, with the protruding elements 15 and gaps 17 residing within the central region 56 of the strut.

As discussed above, according to some implementations the self-expandable member 12 is formed by selectively removing portions of a tube to form the interconnected struts 11 and resultant cell structures 18. As noted above, according to other implementations the self-expandable member 12 is formed by selectively removing portions of a flat sheet to form the interconnected struts 11 and resultant cell structures 18.

When the self-expandable member 12 comprises a laser cut tube, gaps in the form of micro-channels 62 are formed in the tube 60 prior to the laser cutting procedure as shown in FIG. 7A. This may be achieved by tapping the inner surface of the tube 60 to form the micro-channels 62. The tapping tool may comprise cutting elements arranged in a spiral fashion about the outer circumference of the tool. The formation of the micro-channels 62 can thus be achieved by rotating and advancing the tapping tool along a length of the inner lumen of the tube 60. In the implementation of FIG. 7A the micro-channels are provided along substantially the entire length of the inner surface of the tube 60. According to other implementations the cutting elements of the tapping tool are radially deployable and retractable to enable the micro-channels 62 to be formed only at selected locations along the length and/or circumference of the inner surface of the tube 60. In such an implementation when the cutting elements are deployed, the circumference of the cutting elements has a diameter greater than the inner diameter of the tube 60. Conversely, when the cutting elements are retracted, the circumference of the cutting elements has a diameter less than the inner diameter of the tube 60. According to some implementations the cutting elements comprise V-shaped threads located along at least a portion of the length of the tapping tool.

To achieve, for example, the implementation of FIG. 4C, micro-channels 64 may also be formed along the outer surface of the tube 60. According to some implementations the micro-channels 64 are formed by advancing the tube 60 through a die. The die tool may comprise cutting elements arranged in a spiral fashion about the inner circumference of the tool. The formation of the micro-channels 64 can thus be achieved by rotating and advancing the die along a length of the outer surface of the tube 60. In the implementation of FIG. 7B the micro-channels are provided along substantially the entire length of the outer surface of the tube 60. According to other implementations the cutting elements of the die tool are radially deployable and retractable to enable the micro-channels 64 to be formed only at selected locations along the length and/or circumference of the outer surface of the tube 60. In such an implementation when the cutting elements are deployed, the circumference of the cutting elements has a diameter less than the outer diameter of the tube 60. Conversely, when the cutting elements are retracted, the circumference of the cutting elements has a diameter greater than the outer diameter of the tube 60. According to other implementations the micro-channels/gaps 64 are laser cut or etched into the outer surface of the tube 60.

When the self-expandable member 12 is fabricated from a flat metal sheet, gaps in the form of micro-channels may be selectively formed in one or both sides of the metal sheet prior to the sheet being rolled or otherwise formed to assume a substantially tubular configuration. The channels can be formed in a variety of methods including, but not limited to laser cutting, etching, machine cutting, etc.

In the implementations of FIGS. 7A and 7B the cutting elements may comprise an angular orientation that cause the formation of sloping protruding elements 15 as depicted in FIG. 4E.

As mentioned above, the interconnected struts 11 may comprise filaments that are interwoven to form the self-expandable member 12. The filaments may comprise the protruding element 15 and gap 17 features like those illustrated in FIGS. 4A-E. According to some implementations the gaps 17 are formed along a length of the filaments prior to the filaments being interwoven to form the expandable member.

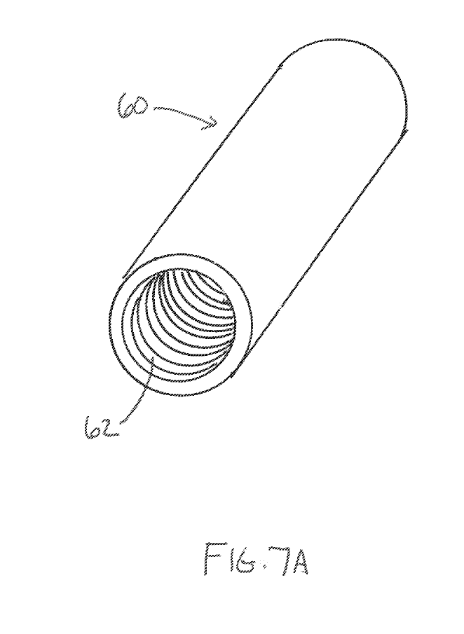

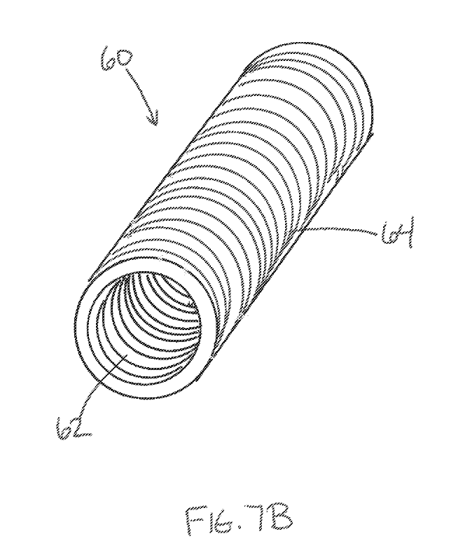

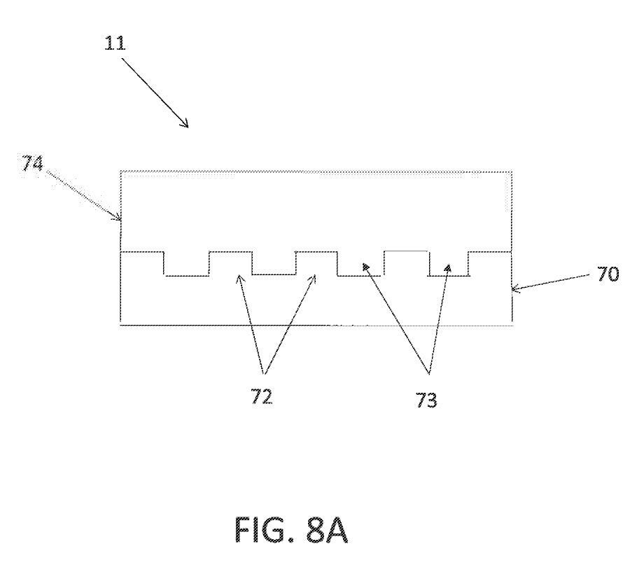

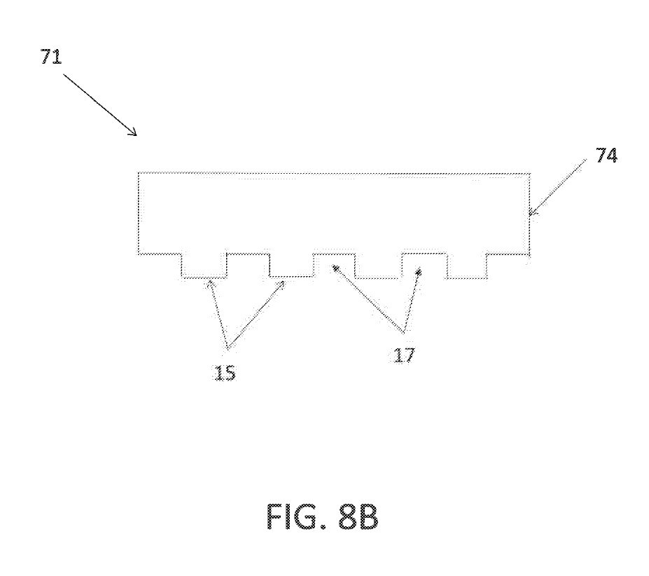

FIGS. 8A and 8B illustrate a process in which the protruding elements 15 and gaps 17 may be formed. In a first step, as shown in FIG. 8A, a layer of metal 74 is deposited onto the surface of a removable substrate 70 using a physical vapour deposition process. The removable substrate 70 comprises protruding features 72 that are separated by gaps 73. Upon the substrate 70 being removed the filament 71 is produced as shown in FIG. 8B having protruding elements 15 and gaps 17 that inversely mimic the geometry of the protruding features 72 and gaps 73 in the removable substrate 70.

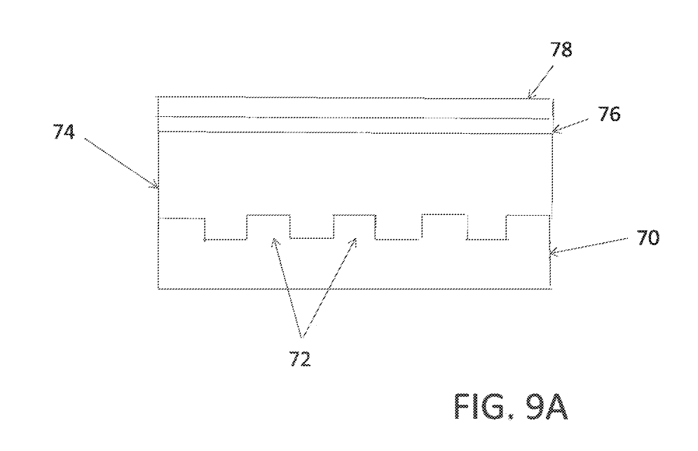

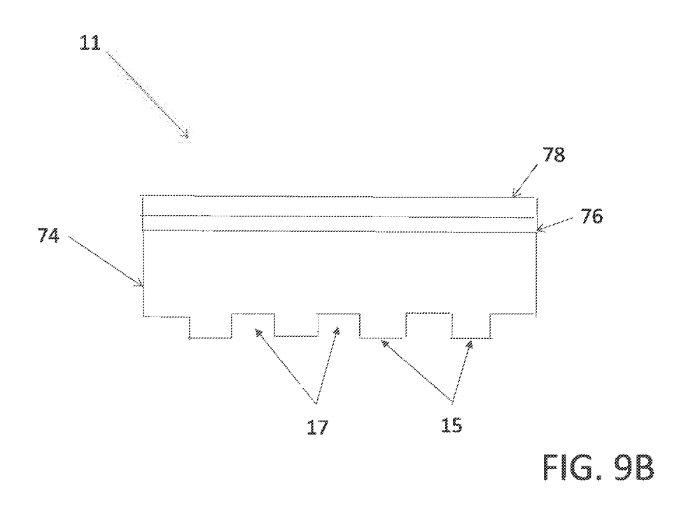

According to other implementations, as shown in FIGS. 9A and 9B, the filament 71 may comprise one or more additional layers formed on the base metal layer 74. For example, at least portions of the outer surface of the base metal layer 74 may have deposited thereon a thin radiopaque layer 76 to assist in fluoroscopically viewing the self-expandable member 12 during its use. The filaments may comprise addition layers, such as a drug eluting layer 78 as depicted in FIG. 9A.

According to other methods, the gaps 17 are laser cut into the filaments before they are interwoven to form the self-expandable member. According to yet other methods the gaps 17 in the surface of the filaments are formed by first masking the surface of the filament so that first portions of the surface of the filament are covered with a resistive layer, such as, for example, a photo-resist layer. Second portions of the surface of the filament are not covered by the resistive layer. Upon the mask being applied to the surface of the filament, the exposed second portions of the filament are chemically etched to form the gaps 17. The masking and etching process occurs prior to the filaments being interwoven to form the expandable member 12.

The methods of FIGS. 8 and 9 described above may be applied to filaments that are interwoven to form the self-expandable member 12, or may applied to a flat metal sheet wherefrom the struts of the self-expandable member are cut or etched. In the latter situation, the method steps may be performed prior to the formation of the struts at the designated locations of at least some or all of the struts. The method steps of FIGS. 8 and 9 may also be performed on at least some or all of the struts after the struts have been formed in the flat metal sheet.

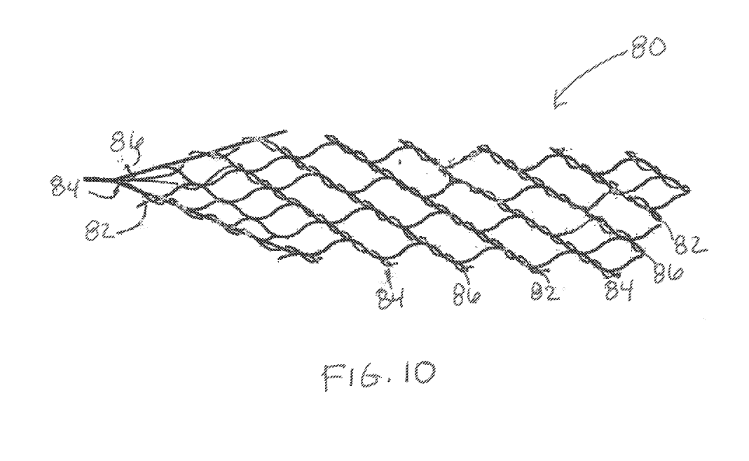

Turning now to FIG. 10, according to other implementations at least some or all of the struts 11 of the self-expandable member 80 are devoid of protruding elements and gaps. Instead, at least some of the interconnected struts 11 are wound about by wires or filaments 82, 84 and 86 having the protruding element 15 and gap 17 features hereunto described. The protruding elements 15 are configured to engage the obstruction/clot as the self-expandable member 80 transition between expanded and unexpanded states. According to some implementations the wires or filaments comprise a radiopaque material.

In the preceding examples for discussion purposes a focus was placed on a procedure for removing blood clots from the neurovasculature of a patient. It is important to note that that the apparatus and methods disclosed herein are not limited to the removal of blood clots, but are applicable to the removal of any penetrable obstruction residing within a bodily duct of a patient.

What is claimed is:

1. A bodily duct obstruction retrieval device comprising:
a self-expandable member that is capable of transitioning between unexpanded and expanded states, the self-expandable member comprising a wall formed by a plurality of interconnected struts, the interconnected struts forming a plurality of cell structures in the wall, the interconnected struts forming the wall having an inner surface and an outer surface, at least some of the interconnected struts having multiple gaps formed in the inner surface, the gaps being separated by protruding elements that are configured to engage the obstruction as the self-expandable member transition from the expanded state toward the unexpanded state, the self-expandable member having a proximal end and a distal end.

2. A bodily duct obstruction retrieval device according to claim 1, wherein protruding elements comprise teeth that are substantially uniformly dispersed along the inner surface.

3. A bodily duct obstruction retrieval device according to claim 2, wherein the teeth are arranged in a saw-tooth configuration.

4. A bodily duct obstruction retrieval device according to claim 1, wherein at least some of the gaps contain a coagulant drug.

5. A bodily duct obstruction retrieval device according to claim 2, wherein the teeth are oriented in a direction facing toward the proximal end of the self-expandable.

6. A bodily duct obstruction retrieval device according to claim 1, wherein only a portion of the inner surface of some of the interconnected struts possess the gaps and protruding elements.

7. A bodily duct obstruction retrieval device according to claim 6, wherein the portions of the inner surface of the interconnected struts that possess the protruding elements are not circumferentially aligned with one another when the self-expandable member is in the unexpanded state.

8. A bodily duct obstruction retrieval device according to claim 6, wherein the outer surface of at least some of the interconnected struts have gaps and protruding elements that are configured to engage the obstruction as the self-expandable member transition from the unexpanded state toward the expanded state.

9. A bodily duct obstruction retrieval device according to claim 1, wherein at least some of the interconnected struts have a first region of a first width and a second region of a second width, the second width being greater than the first width, the gaps and protruding elements residing in the second region and not in the first region.

10. A bodily duct obstruction retrieval device according to claim 9, wherein none of the second regions are circumferentially aligned with one another when the self-expandable member is in the unexpanded state.

11. A bodily duct obstruction retrieval device according to claim 9, wherein the ratio of the second width to the first width is between 1.1 and 1.5.

12. A bodily duct obstruction retrieval device according to claim 1, wherein at least some of the plurality of cell structures comprise a proximal apex region and a distal apex region, the gaps and protruding elements residing within one or both of the proximal and distal apex regions.

13. A bodily duct obstruction retrieval device according to claim 12, wherein the protruding elements comprise teeth that are substantially uniformly dispersed along the inner surface, at least some of the gaps containing a coagulant drug.

14. A bodily duct obstruction retrieval device according to claim 12, wherein none of the proximal and distal apex regions are circumferentially aligned with one another when the self-expandable member is in the unexpanded state.

15. A bodily duct obstruction retrieval device according to claim 1, wherein a radiopaque material is deposited on a surface of the protruding elements.

16. A bodily duct obstruction retrieval device according to claim 2, wherein at least some of the interconnected struts have a first thickness and at least some of the protruding elements have a second thickness, the ratio of the first thickness to the second thickness being between 2.0 and 5.0.

17. A bodily duct obstruction retrieval device according to claim 2, wherein at least some of the interconnected struts have a first thickness and at least some of the protruding elements have a second thickness, the ratio of the first thickness to the second thickness being between 5.0 and 10.0.

18. A bodily duct obstruction retrieval device according to claim 2, wherein at least some of the interconnected struts have a first thickness and at least some of the protruding elements have a second thickness, the ratio of the first thickness to the second thickness being between 15.0 and 60.0.

19. A bodily duct obstruction retrieval device according to claim 1, wherein the interconnected struts are laser cut from a tube or a metal sheet.

20. A bodily duct obstruction retrieval device according to claim 1, wherein the interconnected struts comprise filaments that are interwoven to form the self-expandable member.

21. A bodily duct obstruction retrieval device according to claim 1, further comprising an elongate wire connected with the proximal end of the self-expandable member.
